(19) United States
(12) Patent Application Publication
(10) Pub. No.: US 2025/0325313 A1
(43) Pub. Date: Oct. 23, 2025

(54) ORTHOPAEDIC PLATE AND JIG

(71) Applicant: AT Ortho Limited, Bristol (GB)

(72) Inventors: Roger ATKINS, Bristol (GB); Michael Antony TUKE, Guildford (GB); Andrew Clive TAYLOR, West Sussex (GB)

(21) Appl. No.: 18/870,783

(22) PCT Filed: May 31, 2023
(86) PCT No.: PCT/EP2023/064603

§ 371 Date: Dec. 2, 2024

(30) Foreign Application Priority Data
Jun. 1, 2022 (GB) 2208117.8

(51) Int. Cl. A61B 17/80

(52) U.S. Cl. CPC A61B 17/808; A61B 17/8019

(57) ABSTRACT

The disclosure relates to an assembly for securing an orthopaedic plate to a bone e.g. a tibia. The assembly comprises: an orthopaedic plate having an elongate shaft with bone contact surface configured to contact the bone and an opposing surface. The elongate shaft comprises an array of plate bores, each plate bore for receiving a respective fastener. The assembly further comprises a jig comprising an elongate jig support. The elongate shaft of the plate and the elongate jig support are aligned substantially parallel to each other to define a longitudinally-extending bone-receiving space between the bone contact surface of the elongate shaft and the jig support.

### TECHNICAL FIELD

The present disclosure relates to an orthopaedic assembly comprising an orthopaedic plate and a jig for securing an orthopaedic plate to a bone. In particular, the present disclosure relates to an orthopaedic assembly which may be used, in combination with relevant tools/fasteners, to fix an orthopaedic plate to a patient's bone e.g. to a patient's tibia. In addition, the present disclosure relates to methods of fixing an orthopaedic plate to a bone.

### BACKGROUND

It is known to fix an orthopaedic plate against a patient's bone, e.g. against a patient's tibia, using bone screws in order to reduce and stabilise a fracture within the bone to allow bone healing. The orthopaedic plate is positioned and fixed internally within the patient's limb during orthopaedic surgery optionally with the aid of a jig which is connected to the plate. Such fixations are employed in many sorts of surgery, including fracture treatment, treatment of ununited bones and bone divisions for the correction of deformity.

In the known plate and jig assemblies, the jig support typically extends parallel to the plate, facing and connected to a surface of the plate which, in use faces away from the bone. The plate is inserted against a surgically accessible surface of the bone, which may be superficial underneath the skin, with the plate interposed between the bone and the jig support. Once the plate is in position, fasteners such as bone screws are inserted through bores in the jig support and plate into the bone (from the surgically accessible (e.g. superficial) aspect of the bone) such that the heads of the fasteners engage the plate at the surface of the plate facing the jig support (i.e. facing away from the bone). The bores in the jig support and the plate are aligned to ensure the correct alignment between the fasteners and plate bores.

One problem with the known orthopaedic plates arises as a result of their positioning on the surgically accessible surface of the bone, which may be superficial, just under the patient's skin. This can lead to skin breakdown over the plate which, in extreme cases can lead to limb amputation. Positioning of the plate on a deep surface of the bone i.e. where the bone is interposed between the plate and patient's skin over the superficial surface of the bone should avoid this skin breakdown but these deep bone surfaces are often not accessible by direct surgical exposure. A second issue is that some surgical actions optimally require a plate to be located on a surgically inaccessible or deep bone surface. Such plate positioning is simply not technically feasible using the known plate and jig assemblies. The surgical exposure necessary to position a plate directly on a deep surface of the bone and then to pass the fasteners from deep within the limb, through the plate into the deep surface of the bone would be too great with a significant risk of damage to the nerves and arteries within the limb. It is often possible to place a plate on a bone surface that is not directly surgically accessible by using a short surgical incision at one end of the bone but the fastenings cannot be placed through the plate into the bone in the standard manner detailed above.

The present disclosure has been devised in light of the above considerations with a view to provide a plate and jig assembly that that be used to locate and secure a plate on a deep surface of a bone e.g. the posterior surface of the tibia whilst reducing surgical exposure and risk of nerve/artery damage.

### SUMMARY

In a first aspect, there is provided an assembly for securing an orthopaedic plate to a bone, the assembly comprising: an orthopaedic plate having an elongate shaft with bone contact surface configured to contact the bone and an opposing surface, the elongate shaft comprising an array of plate bores, each plate bore for receiving a respective fastener; and a jig comprising an elongate jig support, characterised in that the elongate shaft of the plate and the elongate jig support are aligned substantially parallel to each other to define a longitudinally-extending bone-receiving space between the bone contact surface of the elongate shaft and the jig support.

By providing an assembly where an orthopaedic plate and jig define, between them, an elongated (longitudinally-extending) space for receiving a bone requiring fixing (e.g. an axially extending fracture portion of bone such as a tibia), the plate can be inserted into the limb (e.g. leg) through a small incision with the elongate shaft positioned against the deep surface of the bone whilst the elongate jig support remains external to the limb such that, in use, the jig can guide relevant fasteners through the bone (from the surgically accessible or superficial surface) and into engagement within the plate bores. Thus, in use, the orthopaedic plate is internal to the limb, the bone contact surface of the elongate shaft contacts the bone, and the elongate jig support is external to the limb. The heads of the fasteners will sit against the surgically accessible surface of the bone. This “transosseous plating” allows positioning of the plate deep within the limb in a surgically inaccessible site and thus does not cause skin breakdown whilst also avoiding the need for any significant surgical exposure and reducing the risk of nerve/artery damage during fastener insertion due to the absence of nerves and arteries proximal the superficial surface of the bone.

Optional features will now be set out. These are applicable singly or in any combination with any aspect or embodiment.

The bone-receiving space between the bone contact surface and the jig support is an unobstructed space prior to insertion into the patient's limb i.e. there are no obstructions extending transversely across the space between the bone contact surface and the jig support so that the bone can positioned within the space without meeting impedance from any part of the assembly. Post insertion (in use), the bone together with a portion of the patient's limb is received within the longitudinally-extending bone-receiving space such that a portion of the limb's skin is interposed between the orthopaedic plate and the jig support, and between the bone and the jig support.

In some embodiments, at least one of the array of plate bores is a threaded plate bore. For example, the or each threaded plate bore may be a conical threaded plate bore having a threaded conical portion tapering from a maximum diameter proximal the bone contact surface to a minimum diameter distal the bone contact surface.

The threaded plate bore(s) e.g. the conical threaded plate bore(s) may (each) comprise a tapered thread having more than one (e.g. two) starts.

The orthopaedic plate of the first aspect may have any one or more of the features described below in relation to the second aspect.

In a second aspect, there is provided an orthopaedic plate for securing to a bone, the plate having an elongate shaft with a bone contact surface configured to contact the bone and an opposing surface, the elongate shaft having an array of threaded bores, each bore for receiving a respective fastener, characterised in that at least one of the array of threaded bores is a conical plate bore having a conical portion tapering from a maximum diameter proximal the bone contact surface to a minimum diameter distal the bone contact surface. In use, the orthopaedic plate is internal to the limb, with the bone contact surface contacting the bone.

The orthopaedic plate of the second aspect is designed for transosseous plating and the plate bores are for receiving the tip portion of a fastener from the bone contact surface. Thus the tapering of the bore from a maximum diameter at the bone contact surface to a reduced diameter provides for the locking of the fastener as the tip portion advances within the conical plate bore from the bone contact surface.

The conical threaded plate bore(s) may (each) comprise a tapered thread having more than one (e.g. two) starts.

In some embodiments, at least one conical plate bore comprises a cylindrical portion (having the maximum diameter) proximal the bone contact surface with a step/shoulder between the cylindrical portion and the conical portion which tapers in diameter away from the cylindrical portion. This cylindrical portion is configured to accommodate a drill bit protruding from the bone without damage to the conical portion. This is discussed in more detail below.

In some embodiments of the first or second aspect, the plate comprises a head portion at an axial end of the elongate shaft. The head portion may have an enlarged width in a direction perpendicular to the elongate shaft and perpendicular to a depth direction between the bone contact surface and the opposing surface. The head portion may comprise at least one head bore. For example, the head portion may comprise a plurality of head bores. The or each head bore may be a threaded head bore. The head bores are provided for each receiving a respective head fastener for affixing the head portion to the enlarged head of the bone e.g. to the enlarged head of the tibia.

The head portion may extend out of the plane of the elongate shaft. For example, the head portion may extend so that there is an angle of greater than 180 degrees between head portion and the bone contact surface and an angle of less than 180 degrees between the head portion and the opposing surface. This allows the elongate shaft to sit against the deep surface of the bone shaft whilst the head portion accommodates the (deep surface of the) enlarged head of the bone.

In the assembly of the first aspect, the head portion may extend at an angle away from the jig support. In the absence of the jig support (e.g. in the second aspect), the angular relationship between the head portion and the surface of the plate may be used to distinguish between the bone contact surface and the opposing surface.

The head portion may comprise a tool receiving portion for receiving an insertion tool. For example, the tool receiving portion may be a recessed portion e.g. a recess or channel for receiving the tool. This facilitates insertion of the plate against the deep surface of the bone as the tool can be engaged in the recessed portion and used to push the elongate shaft within the patient's limb so that the elongate shaft lies against the bone shaft (and the head portion is seated against the enlarged head of the bone).

Insertion of the plate may additionally or alternatively be facilitated by the provision of an insertion portion on the elongate shaft terminating at an insertion axial end (at the opposite axial end to the head portion where provided). The insertion portion may have a rounded insertion axial end. It may be rounded in the plane of the elongate shaft and/or in a plane transecting the elongate plate support in the depth direction between the bone contact surface and the opposing surface.

The insertion portion may taper along the length of the insertion portion to a reduced depth (i.e. to a reduced spacing between the bone contact surface and the opposing surface of the elongate shaft). This may be achieved by tapering of the opposing surface towards the bone contact surface of the elongate shaft.

In the assembly of the first aspect, the opposing surface of the elongate shaft may taper towards the jig support. In the absence of the jig support (e.g. in the second aspect), the tapering of the surface of the plate may be used to distinguish between the surface which is configured to face the bone and the (tapering) opposing surface.

In some embodiments, the opposing surface of the elongate shaft and/or the head portion (i.e. the surfaces configured to face away from the bone) may have a chamfered edge. In the absence of the jig support (e.g. in the second aspect), the location of the chamfered edge may be used to distinguish between the surface which is configured to face the bone and the opposing surface (with the chamfered edge).

The array of plate bores may form a row of bores aligned with the axial extension (length) of the elongate shaft. The plate bores may be aligned with the axis of the elongate shaft. The plate bores may be substantially equally spaced along the length of the elongate shaft.

In some embodiments, the array of plate bores comprises one or more insertion portion bores at or proximal the insertion axial end, wherein at least one of the insertion portion bores is a cylindrical threaded bore. The threaded bore is configured to receive an alignment screw, the purpose of which will be described later.

The jig may comprise a connector portion extending between and rigidly connecting the jig support and the orthopaedic plate. The connector portion is provided to maintain a fixed spatial relationship between the jig support and orthopaedic plate. That is, the connector portion is provided to maintain the elongate jig support and the orthopaedic plate in parallel alignment such that the spacing between the jig support and orthopaedic plate is constant along their respective longitudinal axes. The connector portion may further maintain the elongate jig support and the orthopaedic plate in fixed alignment such that orthopaedic plate overlies the elongate jig support along its longitudinal axis (or vice versa). Conveniently, when the jig support comprises an array of jig bores as discussed below, this can ensure correct alignment of the plate bores with the jig bores. The connector portion is removably connected to the orthopaedic plate. It may be removably connected to the opposing surface of the orthopaedic plate. It may be removably connected to the head portion of the orthopaedic plate e.g. to the opposing surface of the head portion.

The connector portion may comprise a connector body. The connector body may comprise a first socket proximal the orthopaedic plate, the first socket housing a fixing element for removably fixing the connector body to the orthopaedic plate. The opposing surface of the head portion of the plate may comprise locator indents for receiving locator tabs provided on the fixing element. The fixing element may comprise a fixing bore for receiving a connector fixing fastener for removably fixing the fixing element in one of the head bores.

The jig of the first aspect may have any one or more of the features described below in relation to the third aspect.

In a third aspect, there is provided a jig for securing an orthopaedic plate to a bone, the jig comprising an elongate jig support and a connector portion that curves or bends in a plane that is substantially perpendicular to and transects the elongate axis of the jig support.

In the first aspect, the connector portion also curves/bends in a plane that is substantially perpendicular to and transects the elongate axis of the elongate shaft of the orthopaedic plate.

The jig support comprises an internal surface (which, in use, faces the patient's limb) and an opposing external surface. In the assembly of the first aspect, the internal surface faces the bone contact surface of the elongate shaft of the orthopaedic plate i.e. the longitudinally-extending bone-receiving space is defined between the bone contact surface of the orthopaedic plate and the internal surface of the jig support. When the jig comprises the connector portion discussed above, the connector portion is provided to maintain a constant spacing between the internal surface of the jig support and the bone contact surface of the orthopaedic plate along the longitudinal axes of the jig support and the orthopaedic plate. The connector portion may further maintain the elongate jig support and the orthopaedic plate in fixed alignment such that the bone contact surface of the orthopaedic plate overlies the internal surface of the jig support along its longitudinal direction (or vice versa).

In use, the jig support is external to the limb, with the internal surface of the jig support facing the limb.

By providing a connector portion that curves/bends in a plane that is perpendicular to and transects the elongate axis of the jig support/elongate shaft, the connector portion extends laterally from the jig support/elongate shaft leaving the space between the jig support and elongate shaft clear to receive the bone.

In the third aspect, the connector portion may comprise a connector body, first socket and fixing element as described for the first aspect.

The connector portion may be connected to a head of the jig support. In the first aspect, the head of the jig support is substantially aligned with the head portion of the elongate shaft.

The connector body may further comprise a second socket proximal the jig support, the second socket for receiving the head of the jig support. The second socket may span the internal and external surfaces of the head of the jig support.

The connector portion i.e. the connector body may be a single-piece component. The connector portion i.e. the connector body may be substantially C-shaped. The first and second sockets (which may also be C-shaped) are provided at opposing end of the connector body.

In some embodiments of the first or third aspects, the jig support comprises an array of jig bores extending between the internal and external surfaces. The jig bores may be configured to provide unimpeded passage of the respective fasteners through the jig bores when guiding the fasteners through the bone. That is, the jig bores may be configured not to engage with the fasteners when guiding them through the bone. For example, each jig bore may be an unthreaded bore, having a substantially smooth, unthreaded inner surface, such that the jig bore does not engage with a threaded fastener guided therethrough. Additionally, or alternatively, the maximum diameter of each jig bore may be larger than the maximum diameter of each plate bore and/or of each fastener. Each jig bore may be cylindrical.

In the first aspect, the jig bores are each axially aligned with a respective one of the array of plate bores. The axes of the jig/plate bores may be substantially perpendicular to the axis of the elongate plate or may be angularly off-set to accommodate irregular bone profiles.

The jig support of the first or third aspect may also comprise an array of transverse bores on a side surface of the jig support, each transverse bore having an axis perpendicular to the axes of the jig bores and each transverse bore extending from the side surface into a respective one of the jig bores. The purpose of these transverse bores is to secure drill guides within the jig support as will be described in detail below.

In some embodiments of the first or third aspect. the jig support comprises a bone compression device at an opposite axial end of the jig support to the head/connector portion. The bone compression device comprises a compression bore that is slidable towards the head/connector portion. The compression bore has an axis substantially parallel to the axes of the jig bores and is configured to receive a compression screw. The use of the compression screw will be described later.

The compression bore is provided in a compression piece that is slidably mounted within the jig support in a direction aligned with the elongate axis of the jig support. The internal surface of the jig support comprises a slot aligned with the compression piece and exposing the compression bore.

The jig support may further comprise an actuator for effecting sliding of the compression piece within the jig support (and movement of the compression bore/compression screw within the slot on the internal surface of the jig support). The actuator may be, for example, a threaded screw mounted in an adjustment bore at the axial end (opposing the head) of the jig support. The adjustment bore will have an axis parallel with the elongate axis of the jig support. The extension of the threaded screw from the adjustment bore within the jig support can be adjusted by rotation to effect the axial positioning of the compression piece within the jig support and the positioning of the compression bore/compression screw within the slot.

The assembly of the first aspect is used in combination with a number of additional components (e.g. the insertion tool, alignment screw, connector fixing fastener and compression screw previously described) in methods described below to affix the orthopaedic plate to a fractured bone. As described above, the assembly of the first aspect, the orthopaedic plate of the second aspect and the jig of the third aspect can be used in method of transosseous plating where the plate is inserted against a deep surface of the bone with the bone interposed between the plate and the superficial surface of the bone.

Accordingly, in a fourth aspect there is provided an orthopaedic method for securing an orthopaedic plate to a bone within a patient's limb, the plate having an elongate shaft with a bone contact surface and an array of plate bores, the method comprising:
inserting the plate with the elongate shaft against a deep or surgically inaccessible surface of the bone; and
fixing the elongate shaft to the bone by passing a tip portion of a fastener through the bone from a surgically accessible or superficial surface of the bone to engage with a respective one of the plate bores.

The surgically accessible or superficial surface of the bone is generally radially outwards of the surgically inaccessible or deep surface of the bone within the limb proximal the patent's skin.

The plate may be inserted through a percutaneous incision which may be distant from the fracture or bone surgical site. The plate is advanced along the bone with an insertion portion of the plate leading.

After positioning the plate with the bone contact surface against the surgically inaccessible or deep surface of the bone, a head portion of the plate (axially opposed to the insertion portion) may be affixed to the bone. Head fixing fasteners are advanced through head bores in the head portion of the plate from an opposing surface to the bone contact surface and into the deep surface of the bone. This may be done via an appropriate surgical incision.

The method may further comprise (prior to fixing the bone to the elongate shaft) pushing the bone towards the plate by providing a pushing force on the bone (e.g. on the superficial surface of the bone) in a direction perpendicular to the elongate axis of the elongate shaft.

The method may further comprise (prior to fixing the bone to the elongate shaft) pulling the plate towards the bone (i.e. towards the deep surface of the bone) by providing a pulling force on the plate in a direction perpendicular to the elongate axis of the elongate shaft.

It will be clear that the pushing and pull forces described above will be in opposing directions.

In some embodiments, the method may comprise providing a jig having a jig support rigidly connected to the plate. The jig support may be connected to the plate prior to insertion of the against the bone. As the plate is inserted to position the elongate shaft against a deep surface of the bone, the jig support is positioned proximal the surgically accessible or superficial surface of the bone. In this way, the bone is received in a longitudinally-extending space between the elongate shaft and the jig support.

The pushing force referred to above may be applied by providing an array of jig bores in the jig support and inserting at least one pushing element (which may be a bone pusher or may be a drill guide as discussed below) into a respective jig bore to press against the superficial surface of the bone. The extension of the pushing element(s) from the jig bores into the bone-receiving space may be locked by locking fasteners extending through transverse bores in the jig support, each transverse bore extending transversely though the jig support to a respective one of the jig bores to contact a pushing element (e.g. a bone pusher or drill guide) to lock it in place and prevent its movement.

The pulling force may be applied by providing (as part of the array of plate bores) a conical pulling plate bore having a threaded conical portion tapering from a maximum diameter proximal the bone contact surface to a minimum diameter distal the bone contact surface. A pulling screw having a parallel thread matching the minimum diameter of the conical portion may be advanced through the respective jig bore, through the bone from the superficial surface and into the threaded conical pulling plate bore where the intermeshing of the conical and cylindrical bores will pull the plate towards the jig (and thus towards the bone interposed between the plate and jig support).

In some embodiments where the bone fracture requires compression, the method may comprise (prior to fixing the plate to the bone) applying an axial force to the bone in a direction towards the head portion of the plate. This axial force may be applied by a compression screw inserted into the bone (from the superficial bone surface) transverse to the axis of the bone, on an opposing axial side of the fracture to the head portion of the plate, the compression screw being axially slidable in a direction towards the head portion of the plate (which is fixed to the head of the bone). This axial force helps reduce the fracture and align the fractured portions of bone.

Once the fracture or surgical site has been compressed if required using the compression screw and adjustment screw above, the method may comprise further compression of the surgical site by drilling a stabilisation hole through the bone from the surgically accessible bone surface to the deep bone surface, providing a conical stabilisation plate bore (as part of the array of plate bores) having a threaded conical portion tapering from a maximum diameter proximal the bone contact surface to a minimum diameter distal the bone contact surface and passing a stabilisation screw having a parallel thread matching the minimum diameter of the threaded conical portion through the stabilisation hole in the bone from the superficial surface and into the conical stabilisation plate bore where the intermeshing of the conical and cylindrical bores will pull the plate towards the bone and stabilise the compressed fracture. The stabilisation hole through the bone may be provided across the fracture or through a portion of the bone away from the fracture axially opposite the head portion.

In some embodiments, the method comprises providing a cylindrical insertion portion bore (as part of the array of plate bores) and passing an alignment screw through the respective jig bore, through the bone from the superficial surface and into the cylindrical insertion portion bore. This ensures that the elongate shaft of the plate and the jig support remain in alignment.

The step of fixing the plate to the bone by passing the tip portion of the fastener through the bone may comprise drilling a fixing hole through the bone from the surgically accessible or superficial surface to the deep or surgically inaccessible surface.

A plurality of fasteners is preferably used to fix the plate to the bone and thus a plurality of fixing holes will be drilled.

For drilling the fixing hole(s) and/or the stabilisation hole, the method may comprise providing a respective drill guide extending from the jig support (through a respective jig bore) to the surgically accessible or superficial surface of the bone. The drill guide(s) may additionally be used as described above to push on the surgically accessible or superficial surface of the bone to force the bone towards the bone contact surface of the plate. The drill guide(s) are locked in place i.e. their extension from the jig support into the bone-receiving space between the jig support and the plate is fixed using locking fasteners as described above.

The/each drill guide is hollow and defines a drill path for a drill bit from the jig to the bone. The drill bit is kept on the correct drill path (axially aligned with the respective plate bore) as it then advances through the bone to the deep surface of the bone and the bone contact surface of the elongate plate.

The method may comprise controlling the extension of the drill bit from the deep surface of the bone into the plate hole. This may be effected by using a drill stop associated with the drill guide for adjusting the axial length of the drill path (by abutting against the drill and preventing further advancement of the drill bit) and precluding extension into the plate hole beyond a predetermined amount. The extension of the drill bit into the plate hole may be accommodated in a cylindrical portion of the plate hole proximal the bone contact surface of the plate.

The step of fixing the plate to the bone by advancing the tip portion of the fastener(s) through the bone may comprise passing the fastener(s) through the respective jig bore provided in the jig support prior to passing the tip portion(s) through the fixing hole(s) in the bone from the superficial surface.

In some embodiments, method comprises locking the tip portion of the/each fastener in the respective plate bore. This may be achieved by providing (as part of the array of plate bored) a conical plate bore having a threaded conical portion tapering from a maximum diameter proximal the bone contact surface to a minimum diameter distal the bone contact surface. Locking can be effected by using a fastener having a conical tip portion with a thread that matches the thread of the conical portion of the plate bore. Further details of a suitable fastener are described below.

Other methods of locking the tip portion(s) of the fastener(s) in the respective plate bore(s) are also envisaged such as using adhesive.

After fixing the plate to the bone, jig support may be disconnected from the plate.

The method of the fourth aspect may be effected using the assembly of the first aspect.

In a fifth aspect, there is provided an orthopaedic method for securing a plate to a bone using the assembly according to the first aspect, the method comprising:
inserting the plate to position the bone within the longitudinally-extending bone-receiving space between the elongate shaft and the jig support with the bone contact surface of the elongate shaft against the surface of the bone; and
passing the tip portion of a fastener through the bone to engage with a respective one of the plate bores.

In order to position the bone between the plate and jig support and to pass the tip portion of the fastener through the bone (i.e. to effect transosseous plating), the plate will be positioned with the bone contact surface against the surgically inaccessible or deep surface of the bone and the tip portion of the fastener will be passed through the bone from the surgically accessible or superficial surface. The jig support will be provided proximal the surgically accessible or superficial surface of the bone.

The surgically accessible or superficial surface of the bone is generally radially outwards of the surgically inaccessible or deep surface of the bone within the limb proximal the patent's skin.

The method may comprise a first step of connecting the jig to the plate using the connector portion. The locator tabs on the fixing element may be seated in locator indents on the head portion and a connector fixing fastener may be passed through the fixing bore into one of the head bores for fixing the fixing element to the head portion of the plate.

The plate may be inserted through a percutaneous incision which may be distant from the fracture or surgical site. The plate is advanced along the bone with the insertion portion i.e. the insertion axial end of the plate leading. The rounded insertion axial end and/or tapered insertion portion provides for a clean passage of the plate through tissue adjacent the bone.

An insertion tool may be used to push the plate through the tissue. The method may comprise engaging the insertion tool with the tool receiving portion on the head portion of plate. For example, the method may comprise inserting a tool portion into a recess provided on the head portion of the plate.

As the plate is inserted to position the elongate shaft against a deep surface of the bone (e.g. by pushing using the insertion tool), the jig support is positioned proximal the surgically accessible or surface of the bone. In this way, the bone is received in a longitudinally-extending space between the elongate shaft and the jig support.

After positioning the plate with the bone contact surface against the deep surface of the bone, the head portion of the plate (axially opposed to the insertion portion) may be affixed to the bone. Head fixing fasteners are advanced through the head bores in the head portion of the plate from the opposing surface to the bone contact surface and into the deep surface of the bone.

The method may further comprise (prior to fixing the bone to the elongate shaft) pushing the bone towards the plate by providing a pushing force on the bone (e.g. on the surgically accessible or superficial surface of the bone) in a direction perpendicular to the elongate axis of the elongate shaft.

The pushing force may be applied by inserting at least one pushing element into a respective jig bore to press against the superficial surface of the bone. The extension of the pushing element(s) from the jig bores into the bone-receiving space may be locked by locking fasteners extending through the transverse bores in the jig support, each transverse bore extending transversely though the jig support to a respective one of the jig bores to contact a pushing element to lock it in place and prevent its movement.

For example, the method may comprise inserting (and locking) a bone pusher in one of the jig bores e.g. into a jig bore distal the head of the jig support. The bone pusher may be a solid element and may have a pointed end for abutment with the superficial surface of the bone.

The pushing force may additionally or alternatively be provided by inserting (and locking) at least one drill guide extending from the jig support to abut against the superficial surface of the bone. The drill guide may be a hollow member providing a drill path for a drill bit to the bone contact surface of the elongate plate.

The method may further comprise (prior to fixing the bone to the elongate shaft) pulling the plate towards the bone by providing a pulling force on the plate in a direction perpendicular to the elongate axis of the elongate shaft.

The pulling force may be applied by providing (as part of the array of plate bores) a conical pulling plate bore having a threaded conical portion tapering from a maximum diameter proximal the bone contact surface to a minimum diameter distal the bone contact surface. A pulling screw having a parallel thread matching the minimum diameter of the threaded conical portion may be advanced through the respective jig bore, through the bone from the surgically accessible or superficial surface and into the conical pulling plate bore where the intermeshing of the conical and cylindrical bores will pull the plate towards the jig (and thus towards the bone interposed between the plate and jig support).

In some embodiments where the bone fracture requires compression, the method may comprise (prior to fixing the plate to the bone) applying an axial force to the bone in a direction towards the head portion of the plate.

This axial force may be applied using the bone compression device described above for the first and third aspects. The method may comprise passing the compression screw through the compression bore within the compression piece, through the slot in the internal surface of the jig support and into the bone from the surgical accessible or superficial surface transverse to the axis of the bone, on an opposing axial side of the fracture to the head portion of the plate.

The compression device actuator may be used to effect sliding of the compression piece within the jig support towards the head of the jig support. Thus, as the compression piece is moved towards the jig support head, the compression bore and compression screw move within the slot towards the head of the jig support and an axial force (towards the head of the jig support) is applied to the bone via the sliding compression screw. This will reduce and compress the fracture or surgical site in the bone.

Once the fracture or surgical site has been compressed, the method may comprise drilling a stabilisation hole through the bone from the surgically accessible or superficial bone surface to the deep bone surface, providing a conical stabilisation plate bore (as part of the array of plate bores) having a threaded conical portion tapering from a maximum diameter proximal the bone contact surface to a minimum diameter distal the bone contact surface and passing a stabilisation screw having a parallel thread matching the minimum diameter of the threaded conical portion through the stabilisation hole in the bone from the superficial surface and into the conical stabilisation plate bore where the intermeshing of the conical and cylindrical bores will pull the plate towards the bone and stabilise the compressed fracture. The stabilisation hole through the bone may be provided across the fracture or through a portion of the bone away from the fracture axially opposite the head portion.

In some embodiments, the method comprises providing a cylindrical insertion portion plate bore (as part of the array of plate bores) and passing an alignment screw through the respective jig bore through the bone from the superficial surface and into the cylindrical alignment plate bore. This ensures that the elongate shaft of the plate and the jig support remain in alignment.

The step of fixing the plate to the bone by passing the tip portion of the fastener through the bone may comprise drilling a fixing hole through the bone from the superficial surface to the deep surface.

A plurality of fasteners is preferably used to fix the plate to the bone and thus a plurality of fixing holes will be drilled.

For drilling the fixing hole(s) and/or the stabilisation hole, the method may comprise providing a respective drill guide extending from the jig support (through a respective jig bore) to the superficial surface of the bone. The drill guide(s) may additionally be used as described above to push on the superficial surface of the bone to force the bone towards the bone contact surface of the plate. The drill guide(s) are locked in place i.e. their extension from the jig support into the bone-receiving space between the jig support and the plate is fixed using locking fasteners as described above.

The/each drill guide is hollow and defines a drill path for a drill bit from the jig to the bone. The drill bit is kept on the correct drill path (axially aligned with the respective plate bore) as it then advances through the bone to the deep surface of the bone and the bone contact surface of the elongate plate.

The method may comprise controlling the extension of the drill bit from the deep surface of the bone into the plate hole. This may be effected by using a drill stop associated with the drill guide for adjusting the axial length of the drill path (by abutting against the drill and preventing further advancement of the drill bit) and precluding extension into the plate hole beyond a predetermined amount. The extension of the drill bit into the plate hole may be accommodated in a cylindrical portion of the plate hole proximal the bone contact surface of the plate.

The step of fixing the plate to the bone by advancing the tip portion of the fastener(s) through the bone may comprise passing the fastener(s) through the respective jig bore provided in the jig support prior to passing the tip portion(s) through the fixing hole(s) in the bone from the superficial surface.

In some embodiments, method comprises locking the tip portion of the/each fastener in the respective plate bore. This may be achieved by providing (as part of the array of plate bored) a conical plate bore having a threaded conical portion tapering from a maximum diameter proximal the bone contact surface to a minimum diameter distal the bone contact surface. Locking can be effected by using a fastener having a conical tip portion with a thread that matches the thread of the conical portion of the plate bore. Further details of a suitable fastener are described below.

Other methods of locking the tip portion(s) of the fastener(s) in the respective plate bore(s) are also envisaged such as using adhesive.

After fixing the plate to the bone, jig support may be disconnected from the plate.

In order to effect the method of the fifth aspect, there is provided, in a sixth aspect, a kit for securing an orthopaedic plate to a bone, the kit comprising an assembly according to the first aspect and further comprising one or more of the following:
a) a connector fixing fastener for connecting the connector portion of the jig to the plate;
b) an insertion tool for pushing the plate through the tissue along the length of the bone;
c) at least one head fixing fastener for affixing the head portion of the plate to the bone;
d) at least one pushing element such as a bone pusher which may be a solid element and may have a pointed end for abutment with the superficial surface of the bone to push a bone in the bone-receiving space towards the bone contact surface of the elongate plate;
e) a pulling screw having a cylindrical thread matching the minimum diameter of the conical plate bore for extending through a bone in the bone-receiving space into one of the array of plate bores for pulling the elongate shaft towards the bone;
f) a compression screw for extending through the compression bore in the compression device into a bone within the bone-receiving space;
g) a stabilisation screw having a parallel thread matching the minimum diameter of the threaded conical portion of the stabilisation plate bore
h) an alignment screw for extending through a jig bore distal the connector portion and through a bone in the bone-receiving space into the insertion portion bore to ensure alignment of the elongate plate and jig support;
i) at least one fastener having a tip portion for extending through a bone in the bone-receiving space and into one of the array of plate bores for fastening the plate to bone;
j) at least one drill guide for extending through a jig bore to contact a bone within the bone-receiving space, the drill guide for providing a drill path for a drill bit;
k) at least one locking fastener for locking a respective drill bit in the jig support;
l) at least one drill stop for adjusting the axial length of the drill path;
m) at least one drill bit, the/each drill bit having a needle tip.

The or each fastener for fastening the plate to bone preferably has a conical tapered threaded tip portion which matches the threaded conical portion of the respective plate bore (e.g. has the same pitch and/or lead as the respective plate bore). For example, the tip portion may comprise a conical tapered thread having more than one (e.g. two) starts.

The fastener may also have a threaded head portion at an opposing axial end to the tip portion. The thread on the head portion may have a greater pitch than the thread on the tip portion. The head portion may have a greater diameter than the tip portion i.e. the fastener may be tapered along its length from the head portion to the tip portion. The head portion may have a recess in its axial end face for receiving a locking tool for rotating the fastener to advance it through the bone and into engagement with the plate.

The radial spacing between the thread root and thread crest may be greater at the head portion than at the tip portion. The thread crests at the head portion may be sharper than the crests at the tip portion.

The fastener shank (interposed between the tip and head) may also be threaded. The pitch and lead of the shank threads may substantially match the head portion threads. The crests of the shank threads may be less sharp than the head portion threads. When the plate is affixed (fastened) to the bone by the or each fastener, the tip portion of the or each fastener is engaged with a respective plate bore, and the head portion of the or each fastener is engaged directly with a portion of the bone.

The disclosure includes the combination of the aspects and preferred features described except where such a combination is clearly impermissible or expressly avoided.

### SUMMARY OF THE FIGURES

Embodiments will now be discussed with reference to the accompanying figures in which:

FIG. 1 shows a perspective view of the assembly with an attached insertion tool and a bone in situ;

FIG. 2 shows an enlarged view of a conical threaded plate bore;

FIG. 3A shows a sectional view of the assembly with an attached insertion tool and a bone in situ;

FIG. 3B shows an enlarged view of the engagement between the plate and insertion tool;

FIG. 4 shows a perspective view of the assembly with a bone in situ;

FIG. 5 shows a sectional view of the insertion end of the assembly showing the compression device;

FIG. 6a shows a sectional view of the assembly showing inserted drill guides and FIG. 6b shows a perspective view of a drill guide;

FIG. 7A shows the drilled stabilisation hole across the fracture;

FIG. 7B shows a stabilisation screw;

FIG. 8 shows the insertion of the stabilisation screw across the fracture;

FIGS. 9 and 10 show an alignment screw in position within the assembly;

FIG. 11 shows the drilling of a fixing hole using a drill bit;

FIG. 12 shows a locking fastener;

FIG. 13 shows a plurality of locking fasteners in situ within the assembly;

FIG. 14 shows the plate affixed to the bone after removed of the jig; and

FIG. 15 shows a transverse cross section through the lower leg.

### DETAILED DESCRIPTION

Aspects and embodiments will now be discussed with reference to the accompanying figures. Further aspects and embodiments will be apparent to those skilled in the art.

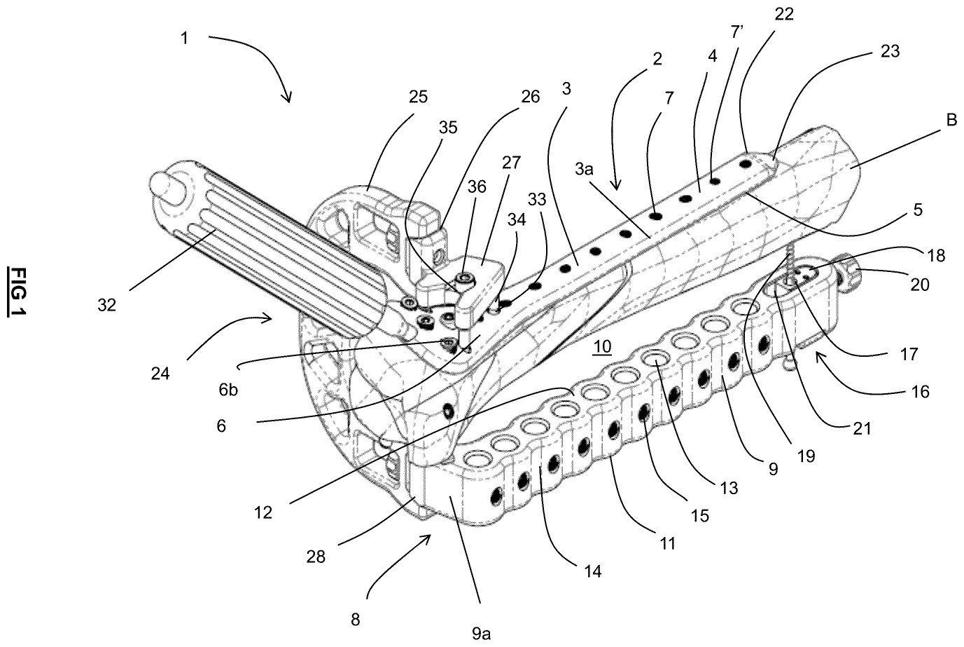

FIG. 1 shows an assembly 1 for securing an orthopaedic plate 2 to a bone B which, in this embodiment is a tibia in a patent's leg. The assembly 1 comprises the orthopaedic plate 2 having an elongate shaft 3 with bone contact surface 5 configured to contact the deep surface of the bone B and an opposing surface 4. The elongate shaft 3 comprises an array of plate bores 7 which form a row of equally spaced bores aligned with the central axis of the elongate shaft 3.

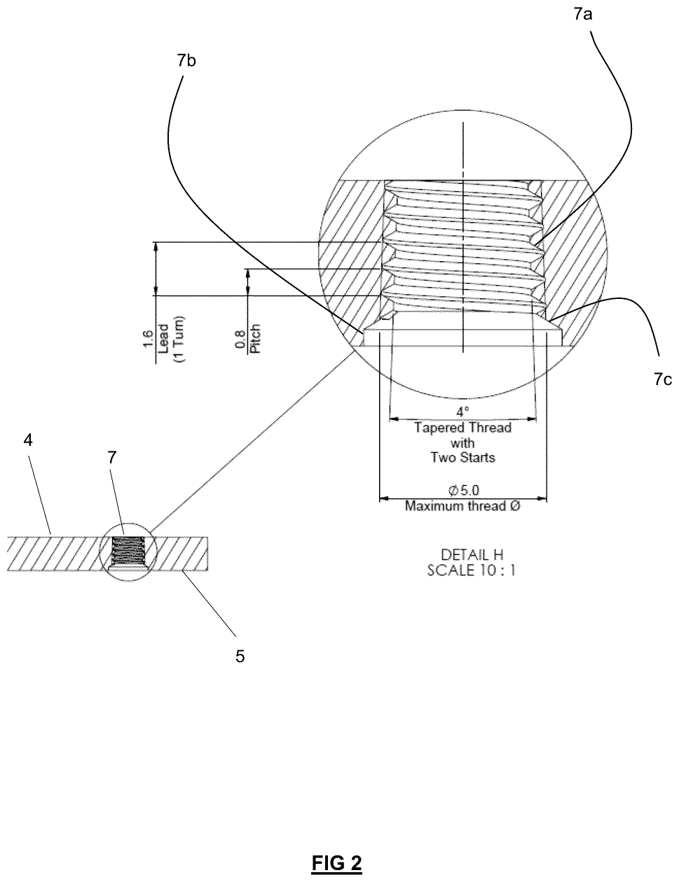

A plate bore 7 is shown in FIG. 2. The plate bore 7 has a threaded portion 7a which is tapered from a maximum diameter of 5 mm proximal the bone contacting surface 5 to a minimum diameter of 4 mm proximal the opposing surface 4. The tapered threaded portion 7a has a pitch of 0.8 mm and has a double start providing a lead of 1.6 mm.

The plate bore 7 has a maximum diameter, unthreaded cylindrical portion 7b at the bone contact surface 5 with a shoulder portion 7c interposed between the tapered threaded portion 7a and cylindrical portion 7b.

The assembly 1 as shown in FIG. 1 further comprises a jig 8 comprising an elongate jig support 9.

The elongate shaft 3 of the plate 2 and the elongate jig support 9 are aligned substantially parallel to each other to define a longitudinally-extending bone-receiving space 10 between the bone contact surface 5 of the elongate shaft 3 and an internal surface 12 of the jig support 9.

The jig support 9 comprises an array of jig bores 13 which extend between the internal surface 12 and an opposing external surface 11.

The axes of the jig bores 13 and plate bores 7 are substantially perpendicular to the elongate axis of the jig support 9 and elongate shaft 3. Opposing jig bores 13 and plate bores 7 (i.e. opposing across the bone-receiving space 10) are axially aligned.

The jig support 9 further comprises a side surface 14 with an array of transverse bores 15. Each transverse bore 15 has an axis perpendicular to the axis of the jig bores 13 and perpendicular to the elongate axis of the jig support 9. Each transverse bore 15 extends from the side surface 14 to join a respective jig bore 13.

The jig support 9 comprises a head 9a at one axial end and a compression device 16 at the opposing axial end. The compression device comprises a compression bore 17 with an axis substantially parallel to the axes of the jig bores 13 that is slidable towards the head 9a of the jig support 9. The compression bore 17 has and is configured to receive a compression screw 19. The use of the compression screw 19 will be described later.

The compression bore 17 is provided in a compression piece 18 that is slidably mounted within the jig support 9 in a direction aligned with the elongate axis of the jig support 9. The internal surface 12 of the jig support 9 comprises a slot 21 aligned with the compression piece 18 and exposing the compression bore 17.

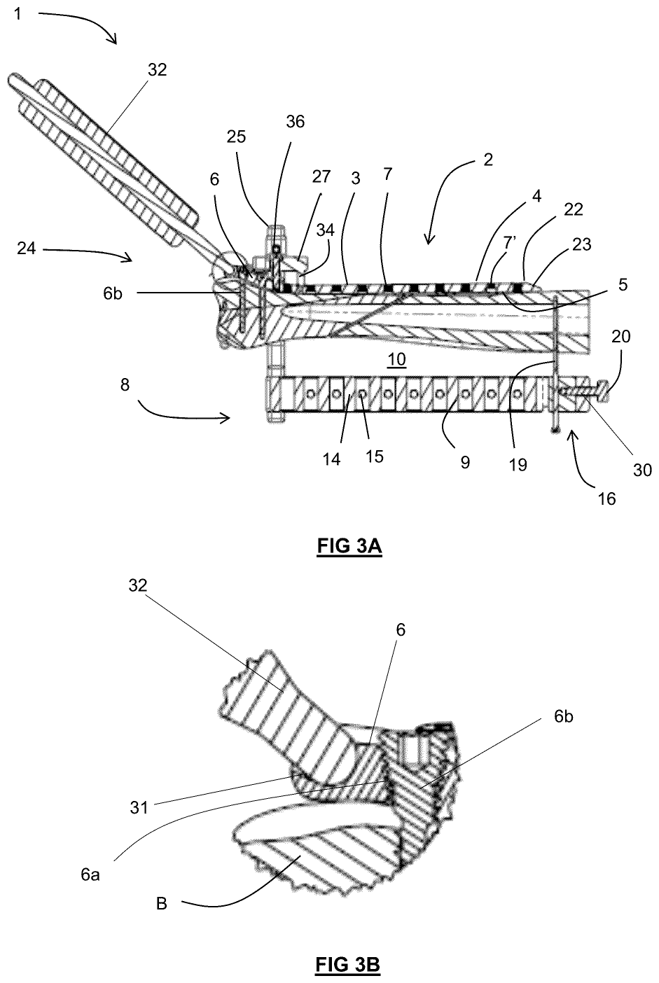

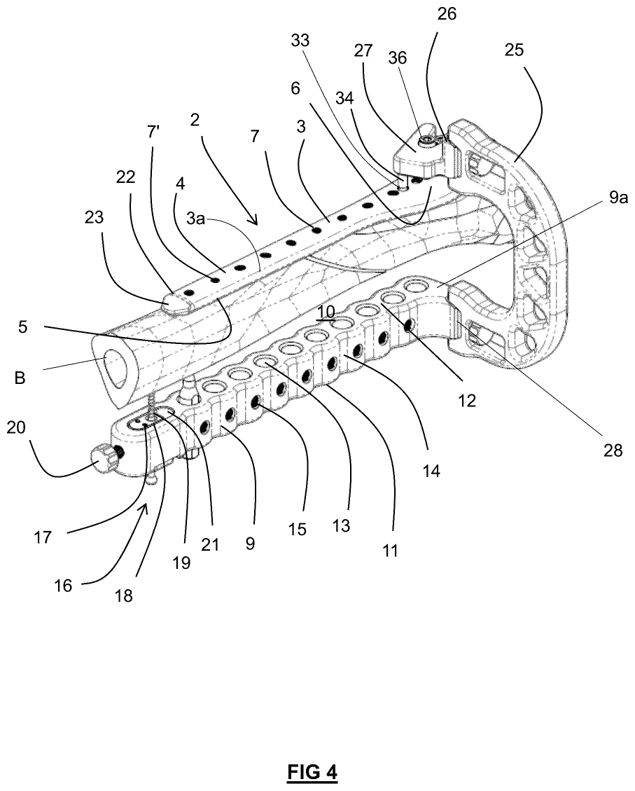

The compression device 16 further comprise an actuator 20 for effecting sliding of the compression piece 18 within the jig support 9 (and movement of the compression bore 18/compression screw 19 within the slot 21 on the internal surface 12 of the jig support 9). The actuator 20 is a threaded screw mounted in an adjustment bore 30 (see FIG. 3A) at the axial end (opposing the head 9a) of the jig support 9. The adjustment bore 30 will have an axis parallel with the elongate axis of the jig support 9. The extension of the threaded screw 20 from the adjustment bore 30 within the jig support 9 can be adjusted by rotation to effect the axial positioning of the compression piece 18 within the jig support 9 and the positioning of the compression bore 18/compression screw 19 within the slot 21.

The plate 2 comprises a head portion 6 at an axial end of the elongate shaft 3. The head portion 6 has an enlarged width in a direction perpendicular to the elongate shaft 3 and perpendicular to a depth direction between the bone contact surface 5 and the opposing surface 4. The head portion 6 may comprises a plurality of threaded head bores 6a.

As is best seen in FIG. 3A for example, the head portion 6 may extend out of the plane of the elongate shaft 3. The head portion 6 extends so that there is an angle of greater than 180 degrees between head portion 6 and the bone contact surface 5 and an angle of less than 180 degrees between the head portion 6 and the opposing surface 4. In this embodiment, the head portion 6 extends by an angle of around 30-40 degrees away from the plane of the elongate shaft 3 (and away from the jig support 9). This allows the elongate shaft 3 to sit against the deep surface of the bone B whilst the head portion 6 accommodates the (deep surface of the) enlarged head of the bone B.

The opposing surface 4 of the elongate shaft 3 and the head portion 6 (i.e. the surfaces configured to face away from the bone B) have a chamfered edge 3a.

The head portion 6 may comprise a tool receiving portion 31 for receiving an insertion tool 32 as shown in FIGS. 3A and 3B. FIG. 3B is an enlarged view of the tool engagement. The tool receiving portion 31 is a recess for receiving the tool 32 and is provided in the axial end of the head portion 6. The tool 32 can be used to facilitate positioning of the plate 2 against the deep surface of the bone B as the tool 32 can be engaged in the recess 31 and used to push the elongate shaft 3 within the patient's limb so that the elongate shaft 3 lies against the bone shaft (and the head portion 6 is seated against the enlarged head of the bone B).

Insertion of the plate 2 is additionally facilitated by the provision of an insertion portion 22 on the elongate shaft 3 terminating at an insertion axial end 23 (at the opposite axial end to the head portion 6). The insertion axial end 23 is rounded in the plane of the elongate shaft 3 and the insertion portion 22 tapers along the length of the insertion portion 22 to a reduced depth (i.e. to a reduced spacing between the bone contact surface 5 and the opposing surface 4 of the elongate shaft 3). This is achieved by tapering of the opposing surface 4 towards the bone contact surface 5 of the elongate shaft 3 (and towards the jig support 9).

The array of plate bores 7 comprises a cylindrical threaded insertion portion bore 7′ proximal the insertion axial end 23. The insertion portion bore 7′ is configured to receive an alignment screw 85, the purpose of which will be described later.

The jig 8 may comprise a connector portion 24 extending between and rigidly connecting the jig support 9 and the orthopaedic plate 2. The connector portion 24 is provided to maintain a fixed spatial relationship between the jig support 9 and orthopaedic plate 2. The connector portion 24 is removably connected to the opposing surface 4 of the head portion 6 of the orthopaedic plate 2.

The connector portion 24 comprises a C-shaped connector body 25 that curves in a plane that is substantially perpendicular to and transects the elongate axis of the jig support 9. By providing a connector portion 24/connector body 25 that curves in a plane that is perpendicular to and transects the elongate axis of the jig support 9/elongate shaft 3, the connector portion 24 extends laterally from the jig support 9/elongate shaft 3 leaving the space 10 between the jig support 9 and elongate shaft 3 clear to receive the bone B.

The connector body 25 comprises a first socket 26 proximal the head portion 6 of the orthopaedic plate 2, the first socket 26 (which is C-shaped in the same plane as the connector body 25) houses a fixing element 27 for removably fixing the connector body 25 to the orthopaedic plate 2. The opposing surface 4 of the head portion 6 of the plate 2 comprises locator indents 33 for receiving locator tabs 34 provided on the fixing element 27. The fixing element 27 comprises a fixing bore 35 for receiving a connector fixing fastener 36 for removably fixing the fixing element 27 in one of the head bores 6a.

The connector body 25 is connected to the plate 2 prior to insertion using the jig assembly 1 for the insertion of the plate 2.

The connector body 25 further comprise a second socket 28 proximal the jig support 9. The second socket 28 has a C-shape and spans the internal and external surfaces 11, 12 of the head 9a of the jig support 9.

The jig assembly 1 is used in methods described below to affix the orthopaedic plate 2 to a fractured bone B.

By providing an assembly 1 where the orthopaedic plate 2 and jig 8 define, between them, an elongated (longitudinally-extending) space 10 for receiving a bone B (e.g. a tibia) requiring fixing, the plate 2 can be inserted into the limb (e.g. leg) through a small incision with the elongate shaft 3 positioned against the deep surface of the bone B whilst the elongate jig support 9 remains external to the limb proximal the superficial surface of the bone B.

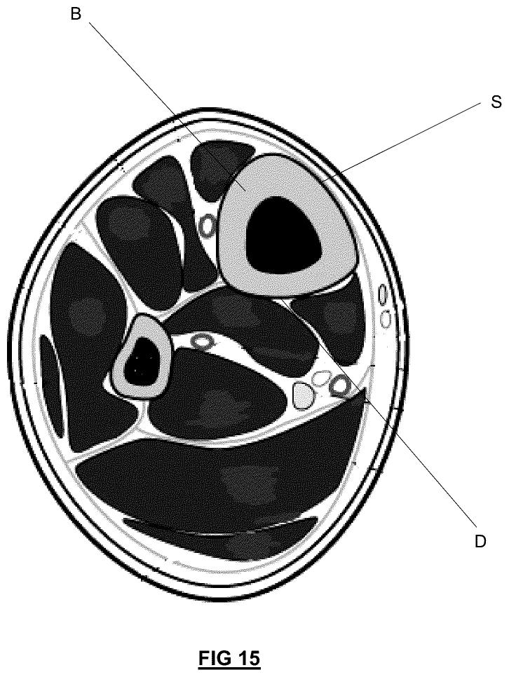

FIG. 15 shows a transverse cross section through the lower leg. The surgically accessible or superficial surface S of the tibia B is adjacent the patient's skin whereas the surgically inaccessible or deep surface D is radially inwards.

The bone-receiving space 10 between the bone contact surface 5 and the jig support 9 is an unobstructed space prior to insertion into the patient's limb i.e. there are no obstructions extending transversely across the space 10 between the bone contact surface 5 and the jig support 9 so that the bone B can positioned within the space 10 without meeting impedance from any part of the assembly 1.

First, the jig 8 is connected to the plate 2 using the connector portion 24. The locator tabs 34 on the fixing element 27 are seated in locator indents 33 on the head portion 6 of the plate 2 and a connector fixing fastener 36 is passed through the fixing bore 35 into one of the head bores 6a to releasably secure the fixing element 27 to the head portion 6 of the plate 2. The connection between the fixing element 27 and the head portion 6 of the plate 2 coupled with the rigidity of the connector portion 24 ensures that the spatial relationship between the elongate shaft 3 and the jig support 9 is maintained.

The plate 2 is inserted through a percutaneous incision which may be distant from the fracture or surgical site F. The plate 2 is advanced along the bone B with the insertion portion 22 i.e. the insertion axial end 23 of the plate 2 leading. The rounded insertion axial end 23 and tapered insertion portion 22 provides for a clean passage of the plate 2 through tissue adjacent the bone B.

The insertion tool 32 is used to push the plate 2 through the tissue. As shown in FIGS. 1, 3A and 3B, the insertion tool 32 is engaged with the tool receiving recess 31 on the head portion 6 of the plate 2.

As the plate 2 is inserted to position (by pushing using the insertion tool 32 until the elongate shaft 3 is against the deep surface D of the bone B), the jig support 9 is positioned proximal the surgically accessible or superficial surface S of the bone B. In this way, the bone B is received in a longitudinally-extending space 10 between the elongate shaft 3 and the jig support 9 as shown in FIGS. 1 and 3A.

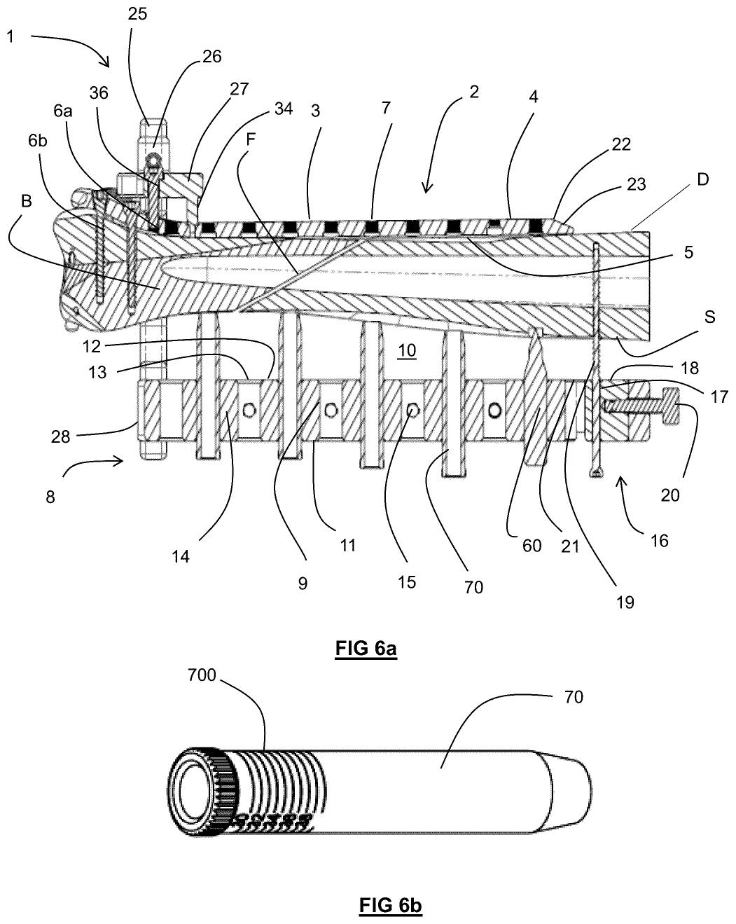

After positioning the plate 2 with the bone contact surface 5 against the surgically inaccessible or deep surface D of the bone B, the head portion 6 of the plate 2 (axially opposed to the insertion portion 22) is affixed to the bone B. Head fixing fasteners 6b are advanced through the head bores 6a in the head portion 6 of the plate 2 from the opposing surface 4 to the bone contact surface 5 and into the deep surface D of the bone B as can be seen in FIGS. 3A and 6, for example.

Prior to fixing the bone B to the elongate shaft 3, the bone B is pushed towards the plate 2 by providing a pushing force on the superficial surface S of the bone B in a direction perpendicular to the elongate axis of the elongate shaft 3.

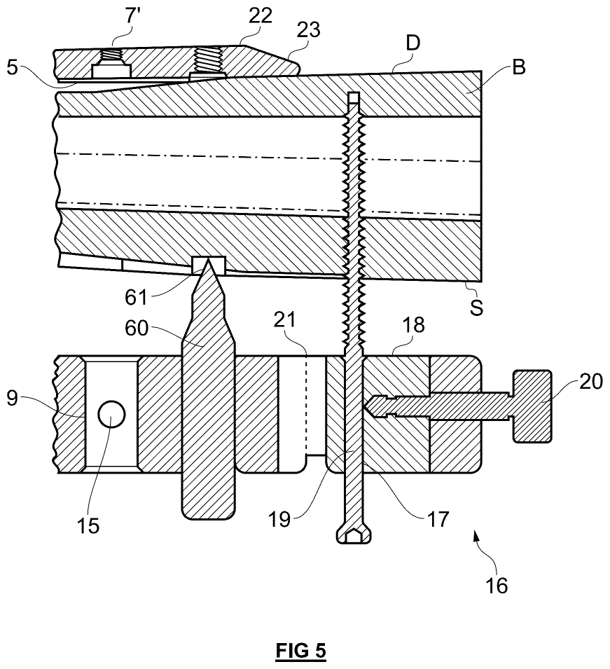

The pushing force is applied by inserting a bone pusher 60 into one of the jig bores 13 distal the head 9a to press against the superficial surface S of the bone B and force it towards the elongate shaft 3. The bone pusher 60 is a solid element with a pointed end 61 for abutment with the superficial surface S of the bone B as shown in FIG. 5.

The bone pusher 60 may be locked in position be inserting a locking fastener (not shown) into the transverse bore 15 associated with the jig bore 13 housing the bone pusher 60 to limit movement of the bone pusher 60 within the jig bore 13.

The pushing force is additionally provided by inserting and locking hollow drill guides 70 extending from the jig support 9 (i.e. extending through a jig bores 13 into the bone-receiving space 10) to abut against the superficial surface S of the bone B as shown in FIG. 6a. These drill guides 70 can be locked in their respective jig bores 13 using locking fasteners (not shown) in the same way as the bone pusher 60. An exemplary drill guide is shown in FIG. 6b. The drill guide 70 has measuring rings 700 to accurately indicate the correct length of the fastener 100.

It can be seen in FIG. 6a that the bone fracture F requires compression. An axial force is applied to the bone in a direction towards the head portion 6 of the plate 2 using the bone compression device 16 described. The compression screw 19 is passed through the compression bore 17 within the compression piece 18, through the slot 21 in the internal surface 12 of the jig support 9 and into the bone B from the superficial surface S transverse to the axis of the bone B, on an opposing axial side of the fracture F to the head portion 6 of the plate 2.

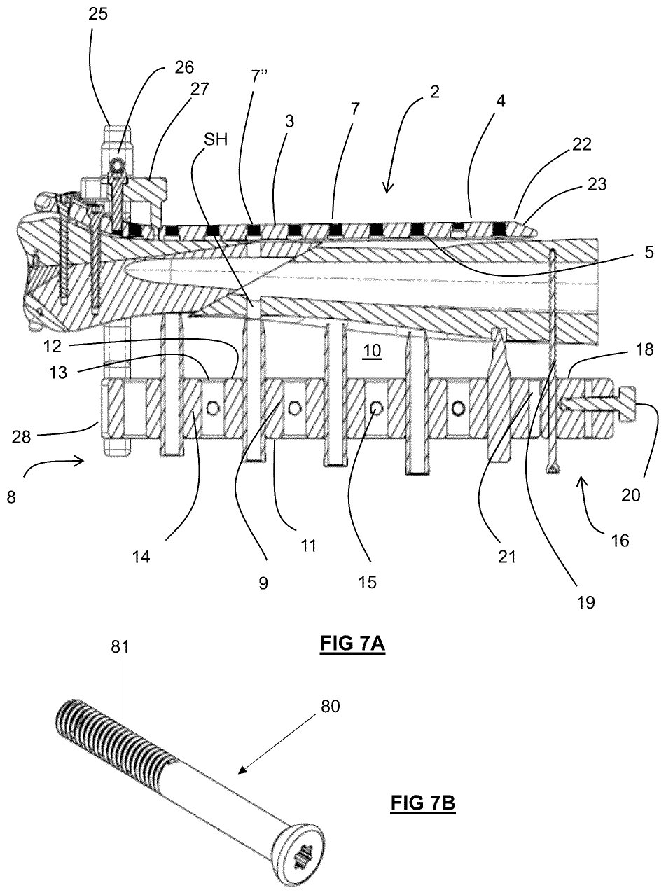

The compression device actuator 20 is used to effect sliding of the compression piece 18 within the jig support 9 towards the head 9a of the jig support 9. Thus, as the compression piece 18 is moved towards the jig support head 9a, the compression bore 17 and compression screw 19 move within the slot 21 towards the head 9a of the jig support 9 and an axial force (towards the head 9 of the jig support 9a) is applied to the bone B via the sliding compression screw 19. This reduces and compresses the fracture F in the bone B as shown in FIG. 7A.

Once the fracture has been compressed, a stabilisation hole SH may be drilled through the bone B across the fracture F from the superficial bone surface S to the deep bone surface D as shown in FIG. 7A. A stabilisation screw 80 such as that seen in FIG. 7B is advanced through the stabilisation hole SH in the bone B from the superficial surface S and into a conical stabilisation plate bore 7″ having a threaded conical portion tapering from a maximum diameter proximal the bone contact surface 5 to a minimum diameter distal the bone contact surface 5. The stabilisation plate bore 7″ has the same dimensions as the plate bores 7.

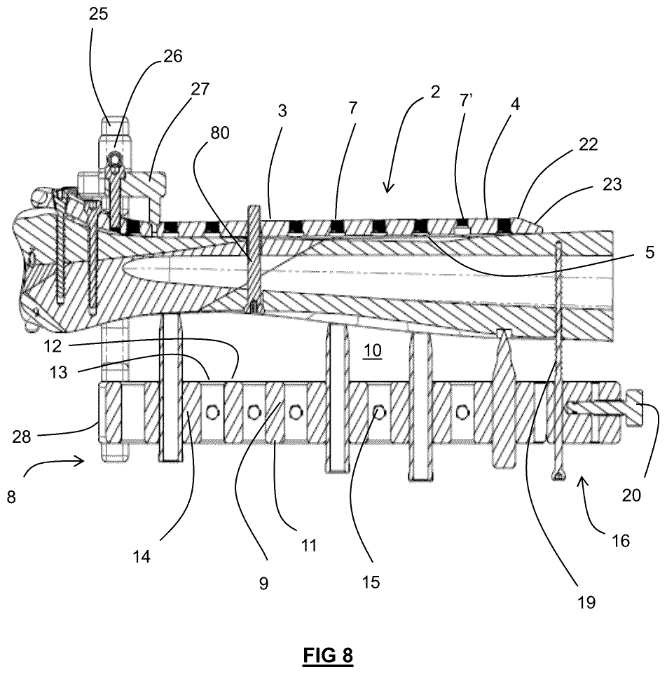

The stabilisation screw 80 has a parallel thread 81 matching the minimum diameter of the threaded conical portion of the conical stabilisation plate bore 7″ and the intermeshing of the conical and cylindrical bores pulls the plate 2 towards the bone B and stabilises the compressed fracture F as shown in FIG. 8.

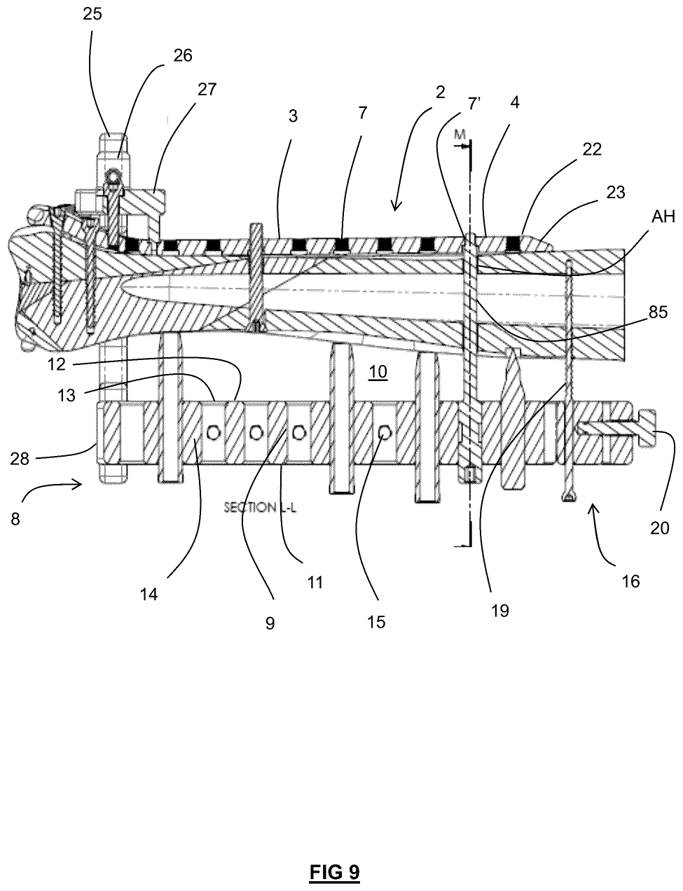

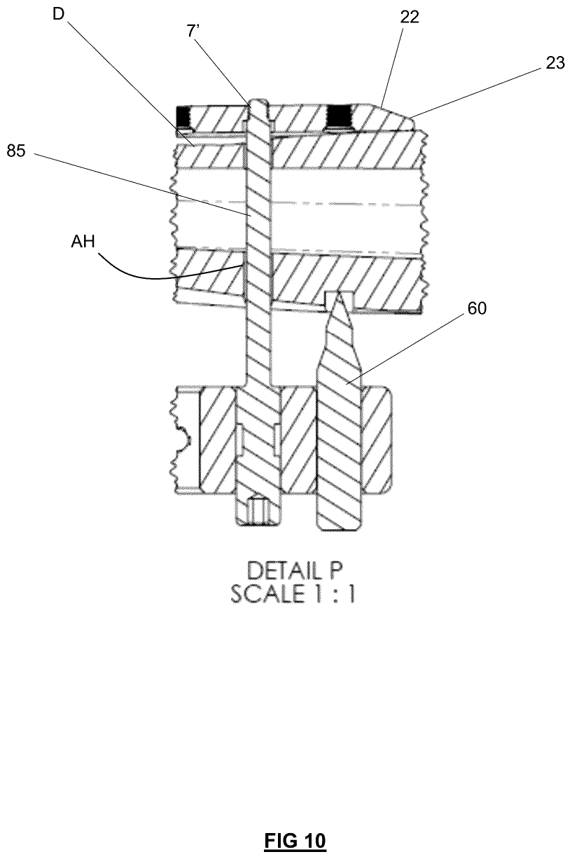

Next, an alignment screw 85 is passed through an alignment hole AH drilled through the bone B and into through the cylindrical insertion portion bore 7′ as shown in FIGS. 9 and 10. This ensures that the elongate shaft of the plate 2 and the jig support 9 remain in alignment.

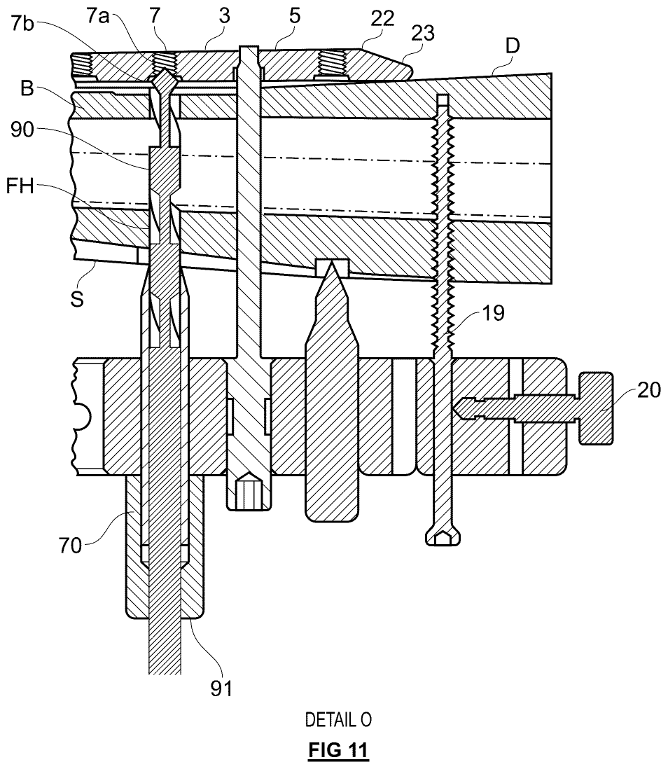

Next, a fixing hole FH is drilled through the bone B from the superficial surface S to the deep surface D as shown in FIG. 11.

For drilling the fixing holes FH, the drill guides 70 (see FIG. 6b) are used to defines a drill path for a drill bit 90 from the jig 8 to the bone B. The measuring rings 700 are used to indicate the correct length of the fastening, The drill bit 90 is kept on the correct drill path (axially aligned with the respective plate bore 7) by the drill guide 70 as it then advances through the bone B to the deep surface D of the bone B and the bone contact surface 5 of the elongate shaft 3.

The extension of the drill bit 90 from the deep surface D of the bone B into the plate hole 7 is controlled using a drill stop 91 which fits over the drill guide 70. The drill stop 91 abuts the external surface 11 of the jig 8 and precludes extension of the drill bit 90 into the plate hole 7 beyond a predetermined amount. The extension of the drill bit 90 into the plate hole 7 may be accommodated in the cylindrical portion 7b of the plate hole 7 proximal the bone contact surface 5 of the plate 2 to avoid any damage to the threaded portion 7a. Further extension of the drill bit 90 is prevented by the drill stop 91 abutting the external surface 11 of the jig 8.

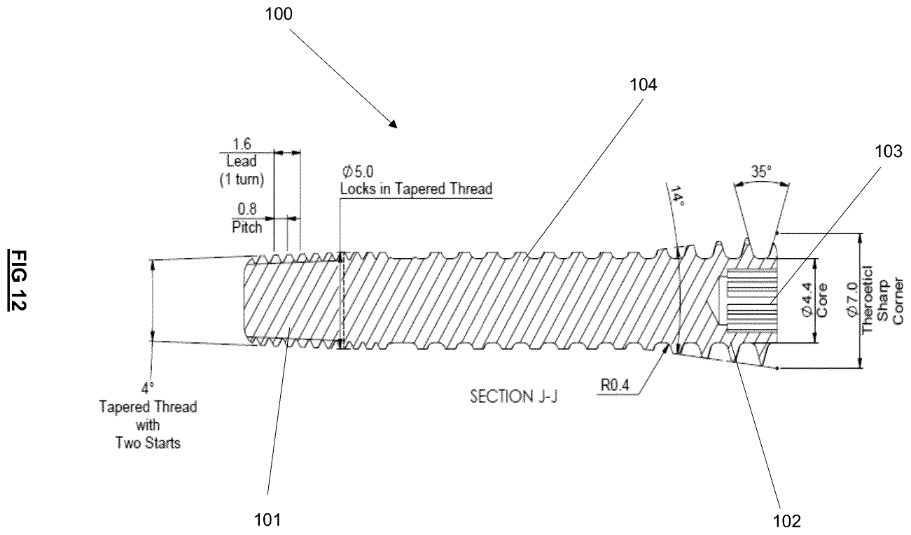

After drilling the fixing holes FH, fasteners 100 such as that shown in FIG. 12 are advanced through the respective jig bore 13 provided in the jig support 9 prior to passing through the fixing hole(s) FH in the bone B from the superficial surface S.

The fastener 100 comprises a conical tapered threaded tip portion 101 which matches the threaded conical portion 7a of the respective plate bore 7 (i.e. has the same pitch (0.8 mm) and lead (1.6 mm) as the respective plate bore 7).

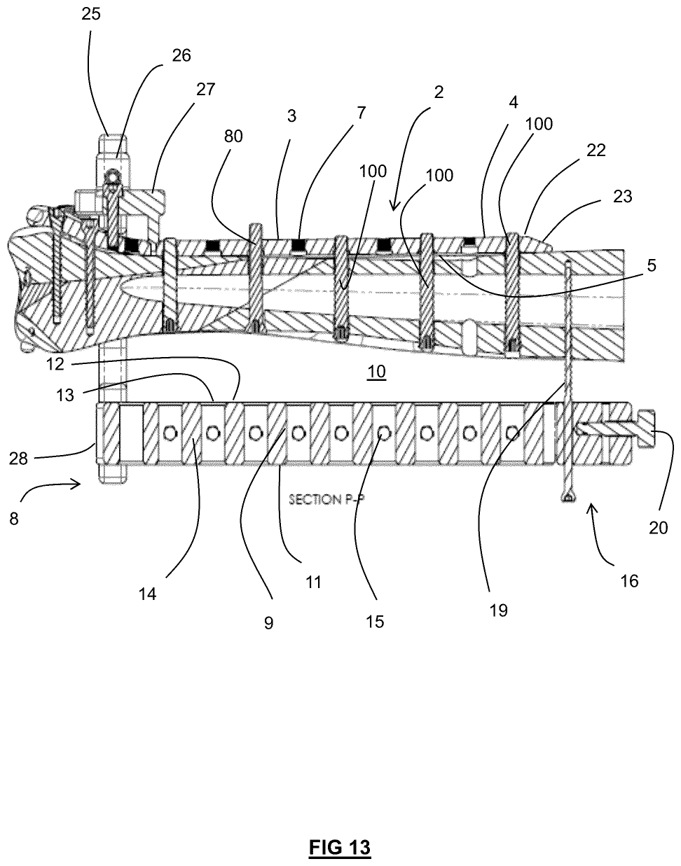

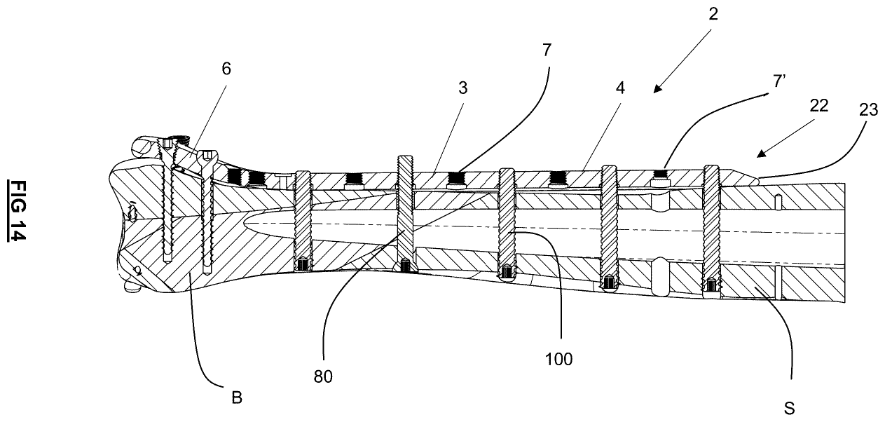

The fastener 100 also has a threaded head portion 102 at an opposing axial end to the tip portion 101. The thread on the head portion 102 has a greater pitch than the thread on the tip portion 101. This allows it to bite into the bone. Thus, the fastener is configured to affix the orthopaedic plate to the bone by engaging its tip portion 101 with a respective plate bore (e.g. by locking the tip portion in the respective plate bore) and by engaging its threaded head portion 102 directly with a portion of the bone (i.e. by biting into the bone). This is illustrated in FIGS. 13 and 14. The head portion has a greater diameter (7 mm) than the tip portion 101 i.e. the fastener 100 is tapered along its length from the head portion 102 to the tip portion 101. The head portion 102 has a recess 103 in its axial end face for receiving a locking tool (not shown) for rotating the fastener 100 to advance it through the bone B in the fixing hole FH and into engagement with the plate 2.

The radial spacing between the thread root and thread crest is greater at the head portion 102 than at the tip portion 101. The thread crests at the head portion 102 are sharper than the crests at the tip portion 101.

The fastener shank 104 (interposed between the tip 101 and head 102) is also threaded. The pitch and lead of the shank threads may substantially match the head portion threads. The crests of the shank threads may be less sharp than the head portion threads.

As the conical tip portion 101 of the fastener 100 engages the respective plate bore 7, the intermeshing of the conical bores effects locking of the tip portion 101 of the fastener 100 in the plate hole 7.

A shown in FIG. 13, a plurality of fasteners 100 are locked into a plurality of plate bores 7.

After fixing the elongate shaft 3 to the bone B, jig support 9 may be disconnected from the plate 2 by detaching the fixing element 27 from the head portion 6 of the plate 2 as shown in FIG. 14.

The heads 102 of the fasteners 100 will sit at the superficial surface S of the bone B. This “transosseous plating” positions the plate 2 deep within the limb and thus does not cause skin breakdown whilst also avoiding the need for any significant surgical exposure and reducing the risk of nerve/artery damage during fastener insertion due to the absence of arteries proximal the superficial surface S of the bone B.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary embodiments described above, many equivalent modifications and variations will be apparent to those skilled in the art. Accordingly, the exemplary embodiments set forth above are considered to be illustrative and not limiting. Various changes to the described embodiments may be made without departing from scope of the claims.

For the avoidance of any doubt, any theoretical explanations provided herein are provided for the purposes of improving the understanding of a reader. The inventors do not wish to be bound by any of these theoretical explanations.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example 15+/−10%.

Further features of the method of the fourth and fifth aspects are set out in the following numbered paragraphs:
1. An orthopaedic method for securing an orthopaedic plate to a bone within a patient's limb, the plate having an elongate shaft with a bone contact surface and an array of plate bores, the method comprising:
   inserting the plate with the elongate shaft against a deep or surgically inaccessible surface of the bone; and
   fixing the elongate shaft to the bone by advancing a tip portion of a fastener through the bone from a superficial or surgically accessible surface of the bone to engage with a respective one of the plate bores.
2. The method of paragraph 1 comprising, prior to fixing the plate, pushing the bone towards the plate by providing a pushing force on the bone in a direction perpendicular to the elongate axis of the elongate shaft.
3. The method of paragraph 1 or 2 comprising, prior to fixing the bone to the elongate shaft, pulling the plate towards the bone by providing a pulling force on the plate in a direction perpendicular to the elongate axis of the elongate shaft.
4. The method of any one of paragraphs 1 to 3 comprising providing a jig having a jig support rigidly connected to the plate and inserting the plate to position the elongate shaft against a deep or surgically inaccessible surface of the bone whilst positioning the jig support proximal the superficial or surgically accessible surface of the bone with the bone received in a longitudinally-extending space between the elongate shaft and the jig support.
5. The method of paragraph 4 comprising, prior to fixing the plate, providing a cylindrical insertion portion bore at an insertion portion of the elongate shaft and passing an alignment screw through the respective jig bore, through the bone from the superficial or surgically accessible surface and into the cylindrical insertion portion bore.
6. The method of any one of paragraphs 1 to 5 comprising, after fixing a head portion of the plate to the bone, applying an axial force to the bone in a direction towards the head portion of the plate.
7. The method of paragraph 6 further comprising drilling a stabilisation hole through the bone from the superficial or surgically accessible bone surface to the deep bone surface, providing a conical stabilisation plate bore (as part of the array of plate bores) having a threaded conical portion tapering from a maximum diameter proximal the bone contact surface to a minimum diameter distal the bone contact surface and passing a stabilisation screw having a parallel thread matching the minimum diameter of the threaded conical portion through the stabilisation hole in the bone from the superficial or surgically accessible surface and into the conical stabilisation plate bore.
8. The method of any one of paragraphs 1 to 7 comprising drilling a fixing hole through the bone from the superficial or surgically accessible surface to the deep or surgically inaccessible surface and advancing the tip portion of a fastener through the fixing hole to engage with a respective one of the plate bores.
9. The method of paragraph 7 or 8 comprising providing a respective drill guide to define a drill path for a drill bit during drilling of the stabilisation and/or fixing holes.
10. The method of paragraph 9 comprising controlling the extension of the drill bit from the deep or surgically inaccessible surface of the bone into the plate hole using a drill stop associated with the drill guide for adjusting the axial length of the drill path.
11. The method of any one of paragraphs 1 to 10 comprising locking the tip portion of the/each fastener in the respective plate bore.
12. The method of any one of paragraphs 1 to 11 using the assembly according to any one of claims 1 to 17.
13. The method of paragraph 12 comprising inserting the plate through a percutaneous incision and advancing the plate along the bone using an insertion tool.
14. The method of paragraph 12 or 13 comprising fixing the head portion to the bone by advancing at least one head fixing fastener through a respective head bore in the head portion of the plate from the opposing surface to the bone contact surface and into the deep or surgically inaccessible surface of the bone.
15. The method of any one of paragraphs 12 to 14 comprising, prior to fixing the plate to the bone, pushing the bone against the bone contact surface of the plate using a bone pusher and/or drill guide extending from a respective jig bore into the bone-receiving space to contact the superficial or surgically accessible surface of the bone.
16. The method of any one of paragraphs 12 to 15 comprising, prior to fixing the plate to the bone by providing a conical pulling plate bore having a threaded conical portion tapering from a maximum diameter proximal the bone contact surface to a minimum diameter distal the bone contact surface, advancing a pulling screw having a parallel thread matching the minimum diameter of the threaded conical portion may be advanced through the respective jig bore, through the bone from the superficial or surgically accessible surface and into the conical pulling plate bore.
17. The method of any one of paragraphs 12 to 16 comprising, prior to fixing the plate, applying an axial force to the bone in a direction towards the head portion of the plate by passing a compression screw through the compression bore within the compression piece, through the slot in the internal surface of the jig support and into the bone from the superficial surface transverse to the axis of the bone, on an opposing axial side of the fracture to the head portion of the plate, using the compression device actuator to effect sliding of the compression piece within the jig support towards the head of the jig support.
18. The method of any one of paragraphs 1 to 17 wherein the bone is a tibia bone in a patient's leg.

1. An assembly for securing an orthopaedic plate to a bone, the assembly comprising:
an orthopaedic plate having an elongate shaft with bone contact surface configured to contact the bone and an opposing surface, the elongate shaft comprising an array of plate bores, each plate bore for receiving a respective fastener; and
a jig comprising an elongate jig support,
wherein the elongate shaft of the plate and the elongate jig support are aligned substantially parallel to each other to define a longitudinally-extending bone-receiving space between the bone contact surface of the elongate shaft and the jig support.

2. An assembly according to claim 1 wherein the jig comprises a connector portion connected between the plate and an axial end of the jig support, the connector portion being curved or bent between the jig support and plate and lying in a plane that is substantially perpendicular to and transects the elongate axis of the jig support.

3. An assembly according to claim 1 wherein the plate comprises a head portion at an axial end of the elongate shaft, the plate comprising at least one head bore, the head portion extending at an angle to the elongate shaft away from the jig support.

4. An assembly according to claim 3 wherein the head portion comprises a tool receiving portion for receiving a tool.

5. An assembly according to claim 1 wherein at least one of the array of plate bores is a threaded plate bore.

6. An assembly according to claim 7 wherein the or each threaded plate bore is a conical plate bore having a conical portion tapering from a maximum diameter proximal the bone contact surface to a minimum diameter distal the bone contact surface.

7. An assembly according to claim 8 wherein the at least one conical plate bore comprises a cylindrical portion proximal the bone contact surface with a shoulder between the cylindrical portion and the conical portion.

8. An assembly according to claim 1 wherein the elongate shaft comprises a tapered insertion portion terminating at an insertion axial end.

9. An assembly according to claim 8 wherein the array of plate bores comprises an insertion portion bore at or proximal the insertion axial end, wherein the insertion portion bore is a cylindrical threaded bore.

10. An assembly according to claim 1 wherein the jig support comprises an array of jig bores on an internal surface facing the bone-receiving space, each jig bore axially aligned with a respective one of the array of plate bores.

11. An assembly according to claim 10 wherein the jig support further comprises an array of transverse bores on a side surface of the jig support, each transverse bore having an axis perpendicular to the axes of the jig bores and each transverse bore extending from the side surface into a respective one of the jig bores.

12. An assembly according to claim 2 wherein the jig support comprises a bone compression device at an opposite axial end of the jig support to the connector portion, the bone compression device comprising a compression bore that is slidable towards the connector portion.

13. An assembly according to claim 12 wherein the compression bore has an axis substantially parallel to the axes of the plate bores and is provided in a compression piece that is slidable within the jig support in a direction aligned with the elongate axes of the plate and jig support.

14. An assembly according to claim 13 further comprising an actuator for sliding the compression piece within the jig.

15. An orthopaedic plate for securing to a bone, the plate having an elongate shaft with a bone contact surface configured to contact the bone and an opposing surface, the elongate shaft having an array of threaded bores, each bore for receiving a respective fastener, and wherein at least one of the array of threaded bores is a conical plate bore having a conical portion tapering from a maximum diameter proximal the bone contact surface to a minimum diameter distal the bone contact surface.

16. A plate according to claim 15 wherein the plate comprises a head portion at an axial end of the elongate shaft, the plate comprising at least one head bore, the head portion extending at an angle to the elongate shaft away from the jig support.

17. A plate according to claim 16 wherein the head portion comprises a tool receiving portion for receiving a tool.

18. A plate according to claim 15 wherein at least one of the array of plate bores is a threaded plate bore.

19. A plate according to claim 15 wherein the at least one conical plate bore comprises a cylindrical portion proximal the bone contact surface with a shoulder between the cylindrical portion and the conical portion.

20. A plate according to claim 15 wherein the elongate shaft comprises a tapered insertion portion terminating at an insertion axial end.

21. A plate according to claim 20 wherein the array of plate bores comprises an insertion portion bore at or proximal the insertion axial end, wherein the insertion portion bore is a cylindrical threaded bore.

22. A jig for securing an orthopaedic plate to a bone, the jig comprising an elongate jig support and a connector portion that curves or bends in a plane that is substantially perpendicular to and transects the elongate axis of the jig support.

23. A jig according to claim 22 wherein the jig support comprises an array of jig bores on an internal surface and an array of transverse bores on a side surface of the jig support, each transverse bore having an axis perpendicular to the axes of the jig bores and each transverse bore extending from the side surface into a respective one of the jig bores.

24. A jig according to claim 22 wherein the jig support comprises a bone compression device at an opposite axial end of the jig support to the connector portion, the bone compression device comprising a compression bore that is slidable towards the connector portion.

25. A jig according to claim 24 wherein the compression bore has an axis substantially parallel to the axes of the plate bores and is provided in a compression piece that is slidable within the jig support in a direction aligned with the elongate axes of the plate and jig support.

26. A jig according to claim 25 further comprising an actuator for sliding the compression piece within the jig.

27. A kit for securing an orthopaedic plate to a bone, the kit comprising an assembly according to claim 1 further comprising one or more of the following:
a) a connector fixing fastener for connecting a/the connector portion of the jig to the plate;
b) an insertion tool for pushing the plate through the tissue along the length of the bone;
c) at least one head fixing fastener for affixing a/the head portion of the plate to the bone;
d) at least one pushing element for abutment with the superficial surface of the bone to push a bone in the bone-receiving space towards the bone contact surface of the elongate plate;
e) a pulling screw having a cylindrical thread for pulling the elongate shaft towards the bone;
f) a compression screw for extending through a/the compression bore in a/the compression device into a bone within the bone-receiving space;
g) a stabilisation screw having a parallel thread for stabilising a fracture;
h) an alignment screw for extending through a jig bore distal the connector portion and through a bone in the bone-receiving space into a/the insertion portion bore to ensure alignment of the elongate plate and jig support;
i) at least one fastener having a tip portion for extending through a bone in the bone-receiving space and into one of the array of plate bores for fastening the plate to bone;
j) at least one drill guide for extending through a jig bore to contact a bone within the bone-receiving space, the drill guide for providing a drill path for a drill bit;
k) at least one locking fastener for locking a respective drill bit in the jig support;
l) at least one drill stop for adjusting the axial length of the drill path;
m) at least one drill bit, the/each drill bit having needle tip.

28. A kit according to claim 27 wherein the tip portion of the/each fastener comprises a respective tapered conical thread.
